(19) United States
(12) Patent Application Publication
(10) Pub. No.: US 2010/0215726 A1
(43) Pub. Date: Aug. 26, 2010

(54) Compositions for Diminishing the Appearance of Wrinkles

(76) Inventor: Peter Thomas Roth, New York, NY (US)

Correspondence Address: MINTZ, LEVIN, COHN, FERRIS, GLOVSKY AND POPEO, P.C, ONE FINANCIAL CENTER, BOSTON, MA, 02111, US

(21) Appl. No.: 12/392,949

(22) Filed: Feb. 25, 2009

(51) Int. Cl. A61K 9/127; A61K 38/08; A61P 17/00

(52) U.S. Cl. 424/450; 514/16

(57) ABSTRACT

Topical skin care compositions are provided for the improvement of the appearance of the skin, namely of the skin comprising the face, surrounding the eyes, mouth, and hands, as well as the skin of the body. The skin care compositions include synergistic combinations of active peptide ingredients that are specially formulated to enhance the natural appearance of the face, hands, and/or body. Specifically, the synergistic skin care compositions may be applied to one or more of the skin surrounding the eyes, mouth, nose, forehead, ears, neck, hands, feed, and overall body to give the skin thereof a cleaner, smoother and more wrinkle free appearance.

### BACKGROUND

Cosmetics may be used to enhance the look of a person's natural appearance. A variety of cosmetic products have been developed to promote the skin's condition and health. For instance, skin care lotions are commonly applied for the purpose of maintaining a healthy, vibrant and youthful appearance. The cosmetic industry has in large part grown to meet the needs of those seeking to make use of such products and practices by promoting and selling makeup and other skin conditioning products that are formulated to enhance the appearance of the skin. An assortment of makeup products to be applied to the body and face are available for sale, these include facial powders, creams, toners, soaps, conditioners and the like, all of which are makeup and/or cosmetic items well known in the art.

One trend currently being followed is to enhance the appearance of the skin of the face, hands, and body. It is commonly thought that having clear, smooth, wrinkle-free skin surrounding the eyes, mouth, hands, and body is aesthetically pleasing. Accordingly, skin care products, such as creams and lotions, are being marketed for their abilities to give a clean and smoother appearance to the skin.

However, many of the products currently sold as cosmetics have only a transitory effect that is limited to the top layers of the skin, i.e., the epidermis. Such cosmetics are meant to be applied and then washed off at a later time. Accordingly, although their use may result in improvement of the appearance, their efficacy is severely limited by the short duration of their effectiveness. Another limitation is that the addition of a cosmetic, if not applied properly, can give a user an unnatural and/or artificial appearance. Current trends in fashion promote a more natural, as opposed to an artificial, appearance. As a result, appearance enhancing products that are designed to promote the body's natural ability to replenish and appear more healthy are highly sought after.

There is, therefore, an interest in developing cosmetic compositions that provide a more natural, healthy and vibrant appearance of the skin and body that is longer lasting, that do not suffer from smearing, and do not give an artificial appearance when used. The compositions and methods of the present disclosure meet these and other needs. Specifically, herein are presented a novel skin care compositions and a method of use thereof that is designed to reverse and/or prevent the damaging effects of free radicals, cleanse and rejuvenate the skin, as well as promote and prolong a more healthy, youthful appearance.

### SUMMARY OF THE DISCLOSURE

Topical skin care compositions are provided for the improvement of the appearance of the skin, namely of the skin comprising the face, surrounding the eyes, mouth, and hands, as well as the skin of the body. The skin care compositions include synergistic combinations of active ingredients that are specially formulated to enhance the natural appearance of the face, hands, and/or body. Specifically, the synergistic skin care compositions may be applied to one or more of the skin surrounding the eyes, mouth, lips, nose, forehead, ears, neck, hands, feet, and overall body to give the skin thereof a cleaner, smoother and more wrinkle free appearance.

The topical skin care compositions of the disclosure include synergistic combinations of one or more natural and/or synthetic proteins, such as one or more of acetyl hexpeptide, palmitoyl tripeptide-3, palmitoyl oligopeptide; palmitoyl tetrapeptide-7, dipeptide diaminobutyroyl benzylamide diacatate; and acetyl glutamyl heptapeptide-1, which compositions are specially formulated to synergistically rejuvenate and condition the skin, as well as to combat the visible effects of aging. For instance, a topical skin care composition of the disclosure may include from about 0.001 to about 20% by weight acetyl hexpeptide, from about 0.001 to about 20% by weight palmitoyl tripeptide-3, from about 0.001 to about 20% by weight palmitoyl oligopeptide and/or palmitoyl tetrapeptide-7, from about 0.001 to about 20% by weight dipeptide diaminobutyroyl benzylamide diacatate, and/or from about 0.001 to about 20% by weight acetyl glutamyl heptapeptide-1.

A topical skin care composition of the disclosure may be formulated for application to the skin, such as the skin surrounding or comprising the eyes, mouth, nose, forehead, ears, neck, hands, feed, and/or overall body. For instance, the topical skin care composition may be formulated as a solution, serum, cream, lotion, body milk, emulsion, balm, and the like. For example, the composition may be formulated as a serum, cream, or balm and formulated for application to the skin around the eyes and/or mouth, to the eyelids, eyebrows, nose, lips, ears, neck, hands, feet, fingers, toes, and the like. In certain embodiments, the skin care composition may be formulated as a powder, e.g., for use as a foundation, and/or application to the face.

Accordingly, the present disclosure features cosmetic skin care compositions that include one or a combination of natural and/or synthetic protein and/or peptide ingredients that function in a synergistic manner to rejuvenate and condition the skin as well as to reduce the appearance of wrinkles therein. In certain variations, the protein and/or peptide compositions include one or more cosmetically acceptable vehicle(s) so as to produce formulations that may be applied topically to the skin in such a manner as to moisturize the surface of the skin, sooth the muscles underlying the surface of the skin, reduce skin irritation and/or inflammation, as well as to promote a more vitalized, healthy, and youthful appearance. The cosmetic formulations of the disclosure may further be combined with one or more vasodilators, anti-inflammatories, or analgesics and/or may additionally include other skin soothing and moisturizing agents.

### DETAILED DESCRIPTION OF THE EMBODIMENTS

The skin is one of the largest organs of the body. In many instances, portions of the skin are continuously exposed to the environment and thus subject to the damaging effects of various environmental factors that are present in the atmosphere. For instance, the skin is often exposed to harmful oxidants, such as UV radiation and other reactive oxygen species, as well as pollutants, harmful chemical agents, microbial, bacterial, and/or viral agents that are present within the environment and which can have damaging effects on the skin. For example, prolonged exposure to UV light, as well as other harmful environmental factors, may lead to the generation of reactive oxygen species within the skin that may harm the natural condition, texture, structure, and/or appearance of the skin. Specifically, exposure to various harmful environmental factors can cause the appearance of aging and wrinkles in an otherwise youthful, smooth portion of skin.

The skin is composed of several layers. Youthful skin is typically pliable, resilient, and thereby poses a stringent barrier to the harmful effects of the environment. The two main layers of the skin are the epidermis and dermis. The dermis lies below the epidermis and is comprised of a number of pores and structural components such as blood vessels, nerves, hair, follicles, smooth muscles, glands, connective tissues and lymphatic tissues.

The outermost layer of the epidermis of the skin is made up of a dense sub-layer of skin called the stratum corneum. The stratum corneum includes about 15 to 20 cell layers that are connected by a small number of adhesive plates, e.g., protein-rich parts of the cell membrane, which give the epidermis both a brick and mortar appearance as well as function. Between the adhesive plates of the stratum corneum are epidermal lipids, which fill the spaces between the cells like mortar or cement, and form a skin-specific lipid barrier in the structure of lamellar bilayer-lipid membranes. The epidermal lipid membranes include about 10 to 30 percent of the total volume of the stratum corneum and pose a permeability barrier which regulates the flow of water and compounds through the skin. Typically, youthful skin has a high degree of keratinization within cells of the stratum corneum as well as a dense packed and cemented configuration of ordered, semicrystalline lipids create a substantially impermeable barrier to penetration.

However, with exposure to adverse environmental agents and/or with the general effects of aging, these proteins and/or epidermal lipids may become degraded and the interstitial fluid between the cells of the skin may decrease in volume leading to a breakdown in the “brick and mortar” configuration of the skin, which in turn, may result in a loss of skin elasticity and the appearance of wrinkles and/or age spots. In such instances, the function of the permeability barrier to both prevent the passage of various substances such as microorganisms, chemicals and allergens, and to minimize transepidermal water loss may be compromised, and thus, with aging, the skin may lose its ability to protect the body from infection and dehydration. Such a compromise may lead to skin that appears rough, old, and wrinkled.

Additionally, although the permeability barrier functions to prevent invasion from foreign molecules, it also functions to prevent and slow the penetration of chemical compounds, such as rejuvenating proteins, peptides, and their analogs, and thereby greatly reduces the effectiveness of cosmetic compositions that employ such proteins and/or their analogs. The longer it takes for an active agent, such as a protein, peptide, and/or its analog, from reaching a site of activity within the layers of the skin, the greater the chances are that the active agent will be broken down and degraded before it has the opportunity to promote its rejuvenating effects within the structures of the skin.

Another barrier to be overcome in the successful infiltration of an active agent, such as a protein and/or peptide composition of the disclosure, into the epidermis and/or into the dermis of the skin is the pH barrier thereof. The effective topical application of many active agents within cosmetic compositions currently available can be difficult because of the insolubility of the active agent(s) in various carrier compositions (such as water, alcohol or oil based carriers) and/or because of the effects that changes in the pH of the skin cause with respect to the permeability barrier of the epidermis. For example, the skin of the face produces a fine oily film that is secreted on to the surface of the skin and has a slightly acidic pH that functions as an integral part of the barrier function of the stratum corneum. The buffer capacity of the skin is the ability of healthy, normal skin to resist a change in pH from acid to alkaline, and the speed at which the skin reacts to an alkaline substance and returns to its natural acid pH value. Youthful skin of the face has a high buffer capacity that actively resists changes in pH. However, with age and/or prolonged exposure to harmful environmental factors the skin may lose its buffer capacity, resulting in increased changes to the pH level of the skin. Changes to the pH level of the skin can cause inflammation, redness and discomfort, all of which should be avoided in a cosmetic composition. Although this pH barrier protects the skin against infection, it also makes the skin less permeable to various agents of the disclosure, especially those active agents that are basic or otherwise polar compounds.

Accordingly, to maximize the effectiveness of a skin care composition, such as a composition including proteins, peptides, and/or their analogs as active agent(s) in a cosmetic composition, the composition may be formulated in such a manner and with agents, active or otherwise, that promote skin penetration while at the same time maintaining and/or modulating the pH of the skin so as to better enable the active agent(s) to deeply penetrate the epidermis and contact the constituents therein. The compositions of the disclosure meet these objectives by presenting novel and synergistic combinations of agents that are specially formulated to rejuvenate the skin, moisturize both the dermis and epidermis, increase the resiliency of the layers of the skin, as well as to relax the underlying muscles thereof. In certain instances, a composition of the disclosure may additionally increase the effectiveness of the barrier of the stratum corneum as well as help restore the pH balance of the buffer capacity of the skin.

A result of the application of a composition of the disclosure to the skin may be a more a healthy, vibrant and youthful appearance. In certain instances, a benefit of the application of a composition of the disclosure to the skin of the body may be a reduction in the appearance of wrinkles, e.g., a substantial reduction in the appearance of wrinkles. By substantial reduction in the appearance of wrinkles is a skin portion that appears to evidence about a 10%, for instance, about a 15%, such as a 20%, for example, a 25%, including a 30% reduction in the appearance of wrinkles as compared to a portion of skin to which a composition of the disclosure has not been applied.

Accordingly, in one aspect of the disclosure, topical skin care compositions are herein provided for the cleansing, conditioning, and/or moisturizing of the skin as well as for rejuvenating and/or preventing damage associated with disruptions caused by harmful exposure to environmental factors (e.g., oxidative stressors) and/or other skin aggravators that may lead to the appearance of aging, e.g., wrinkles, of the skin. The skin care compositions of the disclosure include synergistic combinations of cosmetically effective amounts of various proteins, peptides, analogs thereof, as well as rejuvenating emollient ingredients that are formulated to penetrate the barrier of the skin, moisturize the epidermis and dermis, restore the stratum corneum, rejuvenate the skin's natural elasticity, modulate the endogenous pH balance, and/or prevent or reverse a variety of aging-related conditions of the skin that may lead to the appearance of aging and/or wrinkles within the skin. Accordingly, an effective amount of a topical composition of the disclosure may be used to cleanse and condition the skin as well as to repair and/or protect the skin from various conditions, such as e.g., the appearance of aging, dry skin, dermatitis, eczema, sunburn, inflammation, pruritic lesions, and other inflammatory and non-inflammatory lesions of the skin of a subject. In another embodiment, the present disclosure is to provide cosmetic compositions that may be formulated for topical administration and applied to the skin so as to reduce oxidative stress and/or to rejuvenate the skin.

By a “cosmetically effective amount” (e.g., of a skin care composition of the disclosure) is meant a quantity of the composition provided for topical administration and at a particular dosing regimen that is sufficient to achieve a desired appearance, feel, and/or protective effect. For example, an amount that results in the prevention of or a decrease in the appearance and/or symptoms associated with an undesired condition, such as wrinkles. The amount of the composition to be administered to the subject will depend on the type and severity of the condition of the skin, the amenability of the skin to respond to the formulated agents (e.g., proteins, peptides, and/or their analogs), and on the characteristics of the subject and the subject's natural ability to respond to the synergistic compositions of the disclosure; such characteristics include general health, age, sex, body weight, skin condition, and tolerance to the active agents in the compositions. One of skill will be able to determine appropriate dosages depending on these and other factors.

The term “subject,” as used herein, may be a mammal, such as a human, but may also be an animal, e.g., domestic animals (e.g., dogs, cats and the like), farm animals (e.g., cows, sheep, pigs, horses and the like) and laboratory animals (e.g., rats, mice, guinea pigs and the like).

Without being held to theory, the present cosmetic compositions are based on the discovery by the inventors that the combination of the various proteins, peptides, analogs thereof, and/or emollient agents herein detailed can be formulated into cosmetic topical compositions that are effective in promoting the rejuvenation of the skin, reducing the appearance of aging, and decreasing the amount of damage caused by adverse environmental factors, while at the same time moisturizing and conditioning the skin as compared other such compositions.

It has been found that the active agents of the disclosure are capable of interacting with the skin of the body in such a way as to increase the skin's natural resiliency, moisture content, and self-defense mechanisms. Specifically, without being held to theory, it is believed that the compositions of the disclosure not only promote the structural integrity of the skin, they also moisturize the skin, restore the pH barrier thereof, and act as a barrier in and of themselves to harmful environmental factors. The end result is that a subject's skin that is contacted with the compositions of the disclosure appears to be healthier, more vibrant looking (e.g., evidenced in a decreased appearance of wrinkles), and more resilient when faced with harmful environmental aggravators and/or aging than skin that is not conditioned with the compositions of the disclosure. Accordingly, the compositions of the disclosure create an overall decrease in the environmental stress and moisture loss experienced by the skin and result in a more youthful appearance when applied topically.

Accordingly, one aspect of the present disclosure is to provide rejuvenating cosmetic compositions that include natural and/or synthetic proteins, peptides, and/or their analogs. In another aspect, the rejuvenating compositions of the disclosure may be topically applied to the skin for the rejuvenation thereof. These and other aspects of the present disclosure will be more readily apparent and understood by considering the following description.

### Components of the Cosmetic Compositions

Accordingly, one aspect of the present disclosure is to provide rejuvenating cosmetic compositions that include natural and/or synthetic proteins, peptides, and/or their analogs. In certain embodiments, the rejuvenating compositions of the disclosure may be topically applied to the skin. Specifically, the individual ingredients herein provided have been found to be useful alone or in combination formulation that when applied topically to a portion of the body protects the skin from harmful environmental factors (e.g., such as those that lead to oxidative stress) while also increasing the skin's natural resilience. For instance, in one embodiment, the topical compositions may be applied to the skin so as to clean the skin and give it a more vibrant, youthful appearance and to decrease the indicia of aging, e.g., the appearance of wrinkles, inflammation, abrasions, and lesions.

In certain embodiments, a topical skin care composition of the disclosure may include one or more of (e.g., a synergistic combination of): acetyl hexpeptide, palmitoyl tripeptide-3, palmitoyl oligopeptide, palmitoyl tetrapeptide-7, dipeptide diaminobutyroyl benzylamide diacatate, and/or acetyl glutamyl heptapeptide-1. The active agent(s) of the composition may be formulated in conjunction with a cosmetically acceptable vehicle so as to be applied topically to the skin to clean, rejuvenate, reduce oxidative stress, and/or generally improve the appearance of the skin. The proteins, peptides, and/or analogs found to be useful in accordance with the disclosure are set forth and described herein below.

Acetyl hexapeptide-3, commonly known as ARGIRELINE™ (manufactured by Lipotec, SA and available from Centerchem, Norwalk, Conn.), is a synthetic peptide comprised of the six amino acids: Glutamic Acid-Glutamic Acid-Methionine-Glutamine-Arginine-Arginine (Glu-Glu-Met-Gln-Arg-Arg). Acetyl hexapeptide-3 is an oligopeptide that functions as a catecholamine inhibitor and a muscle relaxant. With out being held to theory, and within the context of the present disclosure, acetyl hexapeptide-3 functions as an agent promoting a reduction in the appearance of wrinkles, for example, by inhibiting the formation of the SNARE complex and/or the release of catecholamines. For instance, the present inventors recognize that acetyl hexapeptide-3 may alleviate muscular contractions when in contact with the muscles present within the dermis and/or epidermis. Specifically, without being held to theory, it is believed that acetyl hexapeptide-3 may reduce the appearance and depth of wrinkles and promote the smoothing of the skin by promoting the relaxation of the muscles and/or nerves that cause contractions within the layers of the skin.

Palmitoyl tripeptide-3 (or Palmitoyl tripeptide-3) is a peptide comprised of 3 amino acids: Glycine-Histidine-Lysine (N-palmitoyl-Gly-His-Lys). Palmitoyl tripeptide may be used individually or in combination with one or more of the peptides described herein. For instance, palmitoyl tripeptide is available as SYN™-COLL (manufactured by Pentapharm, Ltd., Switzerland). Palmitoyl tripeptide is a synthetic peptide that the inventors recognize as a collagen synthesis booster. Without being held to theory, it is recognized that palmitoyl tripeptide may interact with TGF-β so as to promote its interaction with human fibroblasts, which in turn increases the production of collagen thereby protecting and/or restoring the conformation of the collagen triple helix within the tissues of the skin. Specifically, without being held to theory, and within the context of the present disclosure, it is believed that palmitoyl tripeptide promotes collagen production as well as penetration deep within the skin and may also function as a moisturizer thereby promoting a smoother skin that appears less wrinkled.

Palmitoyl oligopeptide (e.g., palmitoyl tripeptide) and/or palmitoyl tetrapeptide-7, and derivatives thereof, commonly known as MATRIXYL™ 3000 (manufactured by Sederma, SA), is a blend of two or more peptide complexes. Palmitoyl oligopeptide (e.g., palmitoyl tripeptide), as set forth above, is a peptide comprised of 3 amino acids: Glycine-Histidine-Lysine (N-palmitoyl-Gly-His-Lys). Palmitoyl tetrapeptide-7 is a peptide comprised of four amino acids: Glycine-Glutamine-Proline-Arginine (N-palmitoyl-Gly-Gln-Pro-Arg). A blend of Palmitoyl oligopeptide (palmitoyl tripeptide) and palmitoyl tetrapeptide-7 when delivered in a mixture functions as a dermal stimulating and/or regenerating composition. The present inventors recognize that a blend of the recited peptides may interact with the tissues of the epidermis and dermis, as well as the matrix inherent in the tissues therein. Further, such a blend may also have the added benefits of promoting penetration within the skin, promoting circulation, acting as an anti-oxidant, for instance, as a metalloprotease inhibitor, and as an anti-inflammatory. Specifically, without being held to theory, and within the context of the present disclosure, it is believed that the recited blend of palmitoyl oligopeptide (palmitoyl tripeptide) and palmitoyl tetrapeptide-7 may interact with the dermal matrix and surrounding tissues to stimulate repair and support of the extracellular matrix of the cells of the skin and thereby reducing the appearance of wrinkles.

Dipeptide diaminobutyroyl benzylamide diacatate, commonly known as SYN™-AKE (manufactured by Pentapharm, Ltd., Switzerland), is a synthetic peptide comprised of the following sequence: H-beta-ala-pro-dab-NHBzl (×2 AcOH). Dipeptide diaminobutyroyl benzylamide diacatate is an oligopeptide that functions as an anti-cholinergic and a muscle relaxant. With out being held to theory, and within the context of the present disclosure, dipeptide diaminobutyroyl benzylamide diacatate functions as an agent that inhibits muscular contraction. For instance, the present inventors recognize that dipeptide diaminobutyroyl benzylamide diacatate may act as a reversible antagonist of nicotinic acetylcholine receptors of the muscles located in and between the epidermis and dermis. Specifically, without being held to theory, it is believed that dipeptide diaminobutyroyl benzylamide diacatate may reduce the appearance of wrinkles and promote the smoothing of the skin by promoting the relaxation of the muscles and/or nerves that cause contractions within the layers of the skin.

Acetyl glutamyl heptapeptide-1 commonly known as SNAP-8 (synaptosome-associated protein—manufactured by Lipotec, SA and available from Centerchem, Norwalk, Conn.), is a peptide comprised of the eight amino acids: Acetyl glutamyl-glutamyl-methionyl-glutaminyl-arginyl-arginyl-alanyl-aspartylamide. Acetyl glutamyl heptapeptide-1 is an oligopeptide that functions at the neuromuscular junction to inhibit nerve cell signals and thereby to relax muscle contractions. With out being held to theory, and within the context of the present disclosure, acetyl glutamyl heptapeptide-1 functions by inhibiting the transmission of neural signals to muscle cells within the skin and thereby diminishing and/or relaxing their contractions. For instance, the present inventors recognize that acetyl glutamyl heptapeptide-1 may act by competing with SNAP-25 at the snap receptor complex to inhibit its formation and thereby destabilize the complex and thus diminish and/or prevent the release of excitatory transmitters. Specifically, without being held to theory, it is believed that acetyl glutamyl heptapeptide-1 may reduce the appearance of wrinkles and promote the smoothing of the skin by promoting the relaxation of the muscles and/or nerves that cause contractions within the layers of the skin.

Accordingly, in a particular embodiment, a composition of the disclosure includes as an active agent at least one or more peptide components selected from (e.g., a synergistic combination of): acetyl hexpeptide, palmitoyl tripeptide-3, palmitoyl oligopeptide, palmitoyl tetrapeptide-7, dipeptide diaminobutyroyl benzylamide diacatate, and/or acetyl glutamyl heptapeptide-1. In certain variations, a composition of the disclosure includes as an active agent one peptide component selected from: acetyl hexpeptide, palmitoyl tripeptide-3, palmitoyl oligopeptide, palmitoyl tetrapeptide-7, dipeptide diaminobutyroyl benzylamide diacatate, and/or acetyl glutamyl heptapeptide-1. In certain variations, a composition of the disclosure includes as an active agent two peptide components selected from (e.g., a synergistic combination of): acetyl hexpeptide, palmitoyl tripeptide-3, palmitoyl oligopeptide, palmitoyl tetrapeptide-7, dipeptide diaminobutyroyl benzylamide diacatate, and/or acetyl glutamyl heptapeptide-1. In certain variations, a composition of the disclosure includes as an active agent three peptide components selected from (e.g., a synergistic combination of): acetyl hexpeptide, palmitoyl tripeptide-3, palmitoyl oligopeptide, palmitoyl tetrapeptide-7, dipeptide diaminobutyroyl benzylamide diacatate, and/or acetyl glutamyl heptapeptide-1. In certain variations, a composition of the disclosure includes as an active agent four peptide components selected from (e.g., a synergistic combination of): acetyl hexpeptide, palmitoyl tripeptide-3, palmitoyl oligopeptide, palmitoyl tetrapeptide-7, dipeptide diaminobutyroyl benzylamide diacatate, and/or acetyl glutamyl heptapeptide-1. In certain variations, a composition of the disclosure includes as an active agent five peptide components selected from (e.g., a synergistic combination of): acetyl hexpeptide, palmitoyl tripeptide-3, palmitoyl oligopeptide, palmitoyl tetrapeptide-7, dipeptide diaminobutyroyl benzylamide diacatate, and/or acetyl glutamyl heptapeptide-1. In certain variations, a composition of the disclosure includes as active agents all of (e.g., a synergistic combination of): acetyl hexpeptide, palmitoyl tripeptide-3, palmitoyl oligopeptide, palmitoyl tetrapeptide-7, dipeptide diaminobutyroyl benzylamide diacatate, and/or acetyl glutamyl heptapeptide-1.

The cosmetic compositions of the present disclosure are formulated for topical use.

Accordingly, in a particular embodiment, a component from the one or more peptides detailed herein may serve as an active agent and may be incorporated into a topical composition by itself or in combination with one or more of the other plant derived active agents. In general, the subject cosmetic compositions contain at least about 0.0001%, at least about 0.01%, at least about 0.1%, or at least about 1.0% and may be as great as or more than about 5% or about 10% or about 15%, or about 20%, or about 25% or more (weight/weight). For instance, in certain variations, a composition of the disclosure may contain from about 0.0001% to about 20% of a peptide selected from one or more of acetyl hexpeptide, palmitoyl tripeptide-3, palmitoyl oligopeptide, palmitoyl tetrapeptide-7, dipeptide diaminobutyroyl benzylamide diacatate, and/or acetyl glutamyl heptapeptide-1 as an active ingredient. In certain variations, a composition of the disclosure may contain from about 0.0001% to about 20% of a peptide selected from all of acetyl hexpeptide, palmitoyl tripeptide-3, palmitoyl oligopeptide, palmitoyl tetrapeptide-7, dipeptide diaminobutyroyl benzylamide diacatate, and/or acetyl glutamyl heptapeptide-1 as an active ingredient.

Without being held to theory, the anti-aging and/or anti-wrinkle effect of the composition of the disclosure stems in part from the synergistic activities of its individual peptide components, which may relax the muscles of the skin, promote the structuring of the collagen and/or extracellular matrix, and/or counteract the oxidative effects of environmental factors, such as free radicals, for instance, reactive oxygen species, which can lead to inflamed, irritated and dry skin and/or promote the generation of other skin-damaging free-radicals. As such, the cosmetic compositions of the present disclosure are uniquely suited to rejuvenate and condition the skin. In some embodiments, the cosmetic compositions of the present disclosure are carefully formulated to provide one or more of acetyl hexpeptide, palmitoyl tripeptide-3, palmitoyl oligopeptide, palmitoyl tetrapeptide-7, dipeptide diaminobutyroyl benzylamide diacatate, and/or acetyl glutamyl heptapeptide-1 in active form as active agents, thereby maximizing the effectiveness of the overall composition.

In many embodiments, additional ingredients are included as part of the subject cosmetic compositions to enhance the synergistic effect of the active agents of the peptide compositions. Accordingly, one or more of the following ingredients may be included in a cosmetic formulation of the present disclosure.

In certain variations, a composition of the disclosure may additionally include one or more other beneficial agents as set forth herein below.

### Skin Soothing/Conditioning Agents

The cosmetic compositions of the present disclosure may also contain agents that sooth, condition and/or rejuvenate the skin. One such agent is panthenol, a pro-vitamin moisturizing agent. Panthenol may be incorporated into cosmetic formulations and may function within a cosmetic composition of the disclosure by readily penetrating the skin to help deliver the active agents to a site of action and to sooth and moisturize the skin. Panthenol derivatives (e.g., ethyl panthenol) may also be used in the compositions of the disclosure as well as other agents such as provitamins B5 and E, as well as their derivatives, aloe vera (e.g., for soothing the skin), allantoin (e.g., for both soothing and softening of the skin), bisabolol (e.g., for soothing the skin), dipotassium glycyrrhizinate, pantothenic acid and its derivatives, as well as Sorbitol (e.g., for moisturizing the skin). A variety of emollients may also be included, such as Neopentyl Glycol Dicaprylate/Dicaprate-ester, Dimethicone, Tridecyl Trimellitate, and other such suitable emollients known in the art. Other skin conditioning/soothing agents can be included in the subject compositions, some of which are discussed below. For instance, one or more of the following may be included: Bis-PEG-18 Methyl Ether Dimethyl Silane, Squalane, PEG 3350, Phospholipids, Vitamin A and its derivatives, Sodium Hyaluronate, Behenyl Alcohol, Butyrospermum Parkii (Shea Butter), Cyclomethicone, Lactobacillus/Eriodictyon Californicum Ferment Extract and the like, Methyl Gluceth-20, Prunus amygdalus dulcis (Sweet Almond) Oil, Sodium Lactate, Borago Officinalis (Borage) Seed Oil, Persea Gratissima (Avocado) Oil, Vitamin D and its derivatives, Caprylic/Capric Triglyceride, and the like. Other beneficial agents include: Lanolin Oil, Lanolin Alcohol, Ceramide III, Urea, Trehalose, mannan, and the like. The skin soothing and/or conditioning agents may be present at a concentration of at least about 0.01% or more, including about 0.25% or more, such about 0.5% or more, for instance about 1.0% to about 10% or more, such as about 20% or more.

### Optional Skin Benefit Materials and Cosmetic Adjuncts

The compositions of the disclosure may optionally include other beneficial materials. These may include steroidiol hormones; progesterone; pregnanalone; coenzyme Q10; methylsolanomethane (MSM); sodium phytate (which may function as a chelating and/or stabilizing agent); copper peptide (copper extract); plankton extract (phytosome); transforming growth factor beta 1 (TGF-β1); glycolic acid; kojic acid; ascorbyl palmitate; all-trans-retinol; azaleic acid; salicylic acid; analgesics; non-steroidal anti-inflammatory drugs (NSAIDs); broparoestrol; and the like. If present, steroids may be present at a concentration of less than about 2% of the total by weight of the composition, while the other optional skin benefit materials may be present at higher levels, for example as much as 10 to 15%.

The compositions may further include sunscreens to lower skin's exposure to harmful UV rays. Sunscreens may include those materials commonly employed to block ultraviolet light. Exemplary compounds are the derivatives of PABA, cinnamate and derivatives of salicylate (other than ferulyl salicylate). For example, octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, Parsol MCX and Benzophenone-3, respectively. Dermascreen may also be used. Additionally, a composition may further include aloe vera or an extracted component or powder thereof, such as anthraquinone glycosides, resins, polysaccharides, sterols, gelonins, and chromones. The exact amount of sunscreen or aloe employed in the compositions can vary depending upon the degree of protection desired from the sun's UV radiation.

The cosmetic compositions of the present disclosure may also contain one or more of the following agents, which agents may act synergistically with the various active agents in various cosmetic compositions of the disclosure to treat aging-related conditions and/or disorders. Butylene Glycol, Saccharomyces Cerevisiae Extract, Tripeptide-29, Sodium Chondroitin Sulfate, Hyaluronic Acid, Silica Dimethyl Silylate, Yeast Polysaccharides, Glycine Soja (Soybean) Seed Extract, Lactobacillus/Capsicum Frutescens Fruit Extract, Pectin, Hydrolyzed Vegetable Protein, Serine, Arginine, Proline. Pentylene Glycol, Sodium Polyacrylate, C12-15 Alkyl Benzoate, Matricaria recutita (Chamomile) Extract, Symphytum Officinale (Comfrey) Extract, Camellia Sinensis (Green Tea) Leaf Extract, Alanine, Ethoxydiglycol, Hydrogenated Lecithin, and the like. Other beneficial materials may include Glycerin, Histidine, Collagen, Elastin, Algae Extract, Echinacea Angustifolia Leaf Extract, Ginkgo Biloba Extract, Vitis vinifera (grape) seed extract, Sodium PCA, Centella Asiatica (Gotu Kola) Extract, Wheat amino acids, Foeniculum vulgare (Fennel) Seed extract, Linoleic acid, Oleic acid, Viola tricolor extract, Rosa Canina Fruit Oil, Superoxide Dismutase, Linoleic acid, Oleic acid, Polyquaternium 51, Sodium cocoyl amino acids, Sarcosine, Potassium aspartate, Magnesium aspartate, Glycosaminoglycans, and the like.

The cosmetic compositions of the present disclosure may also contain benzoyl peroxide, which may act synergistically with the various active agents in various cosmetic compositions of the disclosure to treat aging-related conditions and/or disorders. Benzoyl peroxide is a commonly used topical treatment for mild acne. Benzoyl peroxide has antiseptic properties, i.e., it reduces the number of skin surface bacteria and yeast; is an oxidizing agent, making it keratolytic and comedolytic; and has anti-inflammatory activity. The cosmetic compositions may contain benzoyl peroxide at concentrations contain at least about 1%, at least about 2.5%, at least about 5%, and usually not more than about 10% (weight/weight). Benzoyl peroxide is commercially available as a cream, gel, lotion, or wash under the following brand names: Benoxyl™, Benzac™, Brevoxyl™, Oxy™ and PanOxyl™.

### Cosmetically Acceptable Vehicle

The compositions of the disclosure may include a cosmetically acceptable vehicle to act as a dilutant, dispersant or carrier for the active ingredients, so as to facilitate its distribution and uptake when the composition is applied to the skin. Vehicles other than or in addition to water can include liquid or solid emollients, solvents, humectants, thickeners, powders, and perfumes, some of which have been described above. For instance, a suitable carrier may include, for example, glycerin, butylene glycol, propylene glycol, water, various oils (jojoba, sweet almond, soybean, sunflower, apricot, etc.), and the like.

The cosmetically acceptable vehicle may range form from about 1% or less to about 99.9% or more, such as from about 10% to about 90%, including about 25% to about 80% by weight of the composition, and can, in the absence of other cosmetic adjuncts, form the balance of the composition.

The compositions may be in the form of aqueous, aqueous/alcoholic or oily solutions; dispersions of the lotion or serum type; anhydrous or lipophilic gels; emulsions of liquid or semi-liquid consistency, which are obtained by dispersion of a fatty phase in an aqueous phase (O/W) or conversely (W/O); or suspensions or emulsions of smooth, semi-solid or solid consistency of the cream or gel type. These compositions are formulated according to the usual techniques as are well known to this art.

When the compositions of the disclosure are formulated as an emulsion, the proportion of the fatty phase may range from about 5% or less to about 80% or more by weight, such as from about 10% to about 50% by weight, relative to the total weight of the composition. Oils, emulsifiers and co-emulsifiers incorporated in the composition in emulsion form are selected from among those used conventionally in the cosmetic or dermatological field. The emulsifier and coemulsifier may be present in the composition at a proportion ranging from about 0.3% or less to about 30% or more by weight, such as from about 0.5% to about 20% by weight, relative to the total weight of the composition.

When the compositions of the disclosure are formulated as an oily solution or gel, the fatty phase may constitute more than about 50% or less, more than about 60%, more than about 70%, more than about 80%, more than about 90% of the total weight of the composition.

The compositions of the disclosure may be in the form of body cleansing compositions. As such, these compositions may contain one or more wash-active surfactant in an aqueous base. The surfactants can be present, alone or in a mixture, and are contained in an amount of from about 1% or less to about 50% or more by weight or from about 1% or less to about 30% or more by weight. Nonionic surfactants, amphoteric surfactants, zwitterionic surfactants and anionic surfactants are generally suitable.

Suitable anionic surfactants include, e.g. alkaline or alkaline earth salts, alpha-olefin sulfonates, sulfosuccinates, disodium laureth-3 sulfosuccinate, disodium PEG-5 lauryl citrate sulfosuccinate, disodium ricinolamido MEA-sulfosuccinate or disodium laurylamido MEA-sulfosuccinate and alkyl ether carboxylates.

Suitable nonionic surfactants include e.g., alkoxylated fatty alcohols, alkoxylated fatty acid esters, alkoxylated partial glycerides, saturated or unsaturated fatty acids, alkoxylated polyol esters, and alkylpolyglucosides, such as coconut glucosides, lauryl glycosides or decylglucosides. For example, ethoxylated lauryl alcohol, tetradecyl alcohol, cetyl alcohol, oleyl alcohol or stearyl alcohol, which are used alone or in mixtures with each other, as well as fatty alcohols of ethoxylated lanolin, are suitable as fatty alcohol ethoxylates. Furthermore the ethoxylated fatty acid sugar esters known as nonionic surfactants, especially ethoxylated sorbitan fatty acid ester, are suitable for use in the cosmetic preparations according to the disclosure. The suitable ethoxylated fatty acid sugar esters include those marketed under the trade names Tween™ and Arlacel™ by ICI surfactants and the alkyl-polyglycosides, which are marketed under the trade names Plantaren™ or Plantacare™ by Henkel or under the trade name Oramix™ by Seppic. Suitable amphoteric surfactants include for example betaines, such as cocoamidopropylbetaine or lauryl betaine, sulfobetaines, such as cocoamidopropyl hydroxysultaine, glycinates, such as cocoamphoglycinate (INCI-name: sodium cocoamphoacetate) and diglycinates and propionates, such as cocoampho-propionate.

The compositions of the disclosure may also include additives and adjuvants which are conventional in the cosmetic, pharmaceutical or dermatological field, such as hydrophilic or lipophilic gelling agents, hydrophilic or lipophilic active agents, preservatives, antioxidants, solvents, fragrances, perfumes, sunscreens, fillers, bactericides, odor absorbers and dyestuffs or colorants. The amounts of these various additives and adjuvants are those conventionally used in the field, and, for example, range from about 0.01% or less to about 10% or more of the total weight of the composition. Depending on their nature, these additives and adjuvants may be introduced into the fatty phase or into the aqueous phase.

Exemplary oils which may be used according to this disclosure include mineral oils (liquid petrolatum), plant oils (liquid fraction of karite butter, sunflower oil), animal oils (perhydrosqualen(e), synthetic oils (purcellin oil), silicone oils (cyclomethicone) and fluoro oils (perfluoropolyethers). Fatty alcohols, fatty acids (stearic acid) and waxes (paraffin wax, carnauba wax and beeswax) may also be used as fats.

Emulsifiers which may be used include glyceryl stearate (e.g., to help the oil and water phases combine), polysorbate 60, PEG-6/PEG-32/glycol stearate mixture, etc. Solvents which may be used include the lower alcohols, in particular ethanol and isopropanol, and propylene glycol. Suitable fatty alcohols and acids include those compounds having from 10 to 20 carbon atoms. For instance, compounds such as cetyl, myristyl, palmitic and stearyl alcohols and acids. For example, cetearyl alcohol and ceteareth 20, as well as cetyl alcohol and stearyl alcohol-fatty alcohols may also be included.

Hydrophilic gelling agents include carboxyvinyl polymers (carbomer), acrylic copolymers, such as acrylate/alkylacrylate copolymers, polyacrylamides, polysaccharides, such as hydroxypropylcellulose, natural gums and clays (such as Xanthan gum), and, as lipophilic gelling agents, representative are the modified clays such as bentones, fatty acid metal salts such as aluminum stearates and hydrophobic silica, or ethylcellulose and polyethylene.

An oil or oily material may be present, together with an emollient to provide either a water-in-oil emulsion or an oil-in-water emulsion, depending largely on the average hydrophilic-lipophilic balance (HLB) of the emollient employed. Levels of such emollients may range from about 0.5% to about 50%, preferably between about 5% and 30% by weight of the total composition. Emollients may be classified under such general chemical categories as esters, fatty acids and alcohols, polyols and hydrocarbons.

Esters may be mono- or di-esters. Acceptable examples of fatty di-esters include dibutyl adipate, diethyl sebacate, diisopropyl dimerate, and dioctyl succinate. Acceptable branched chain fatty esters include 2-ethyl-hexyl myristate, isopropyl stearate and isostearyl palmitate. Acceptable tribasic acid esters include triisopropyl trilinoleate and trilauryl citrate. Acceptable straight chain fatty esters include lauryl palmitate, myristyl lactate, oleyl eurcate and stearyl oleate. Preferred esters include coco-caprylate/caprate (a blend of coco-caprylate and coco-caprate), propylene glycol myristyl ether acetate, diisopropyl adipate and cetyl octanoate.

Among the polyols which may serve as emollients are linear and branched chain alkyl polyhydroxyl compounds. For example, propylene glycol, sorbitol and glycerin are preferred. Also useful may be polymeric polyols such as polypropylene glycol and polyethylene glycol. Butylene and propylene glycol are also especially preferred as penetration enhancers.

Exemplary hydrocarbons which may serve as emollients are those having hydrocarbon chains anywhere from 12 to 30 carbon atoms. Specific examples include mineral oil, petroleum jelly, squalene and isoparaffins.

Another category of functional ingredients within the cosmetic compositions of the present disclosure are thickeners. A thickener may be present in amounts anywhere from about 0.1% or less to about 20% or more by weight, such as from about 0.5% to about 10% by weight of the composition. Exemplary thickeners may be cross-linked polyacrylate materials available under the trademark Carbopol. Gums may be employed such as xanthan, carrageenan, gelatin, karaya, pectin and locust beans gum. Under certain circumstances the thickening function may be accomplished by a material also serving as a silicone or emollient. For instance, silicone gums in excess of 10 centistokes and esters such as glycerol stearate have dual functionality.

Powders may be incorporated into the cosmetic composition of the disclosure. These powders include chalk, talc, kaolin, starch, smectite clays, chemically modified magnesium aluminum silicate, organically modified montmorillonite clay, hydrated aluminum silicate, fumed silica, aluminum starch octenyl succinate and mixtures thereof.

Other adjunct minor components may also be incorporated into the cosmetic compositions. These ingredients may include coloring agents, opacifiers and perfumes. Amounts of these other adjunct minor components may range anywhere from about 0.001% or less up to about 20% or more by weight of the composition.

### Product Use, Form, and Packaging

In use, a quantity of the composition, for example from about 1 ml or less to about 100 ml or more, is applied to a site of interest (e.g., skin, scalp, etc.) from a suitable container or applicator and, if necessary, it is then spread over and/or rubbed into the site using the hand or fingers or a suitable device, such as a brush, comb, or other suitable applicator. The composition may be specifically formulated for use as a treatment for a specific area, e.g. the hands, the face, the scalp, the feet, etc.

The cosmetic composition of the disclosure may be formulated in any form suitable for application to the site of interest, including a lotion, cream, gel, shampoo, etc. The composition can be packaged in a suitable container to suit its viscosity and intended use by the consumer. For example, a lotion or cream can be packaged in a bottle, or a propellant-driven aerosol device or a container fitted with a pump suitable for finger operation. When the composition is a cream, it can simply be stored in a non-deformable bottle or squeeze container, such as a tube or a lidded jar. The disclosure accordingly also provides a closed container containing a cosmetically acceptable composition as herein defined.

The following examples are put forth so as to provide those of ordinary skill in the art with a complete disclosure and description of how to make and use the subject compositions, and are not intended to limit the scope of what is regarded as the disclosure. Efforts have been made to insure accuracy with respect to the numbers used (e.g. amounts, temperature, concentrations, etc.) but some experimental errors and deviations should be allowed for. Unless otherwise indicated, parts are parts by weight, molecular weight is weight average molecular weight, temperature is in degrees centigrade, and pressure is at or near atmospheric.

### Example 1

Example 1 illustrates topical compositions according to the present disclosure. The compositions can be processed in accordance with conventional manners well known in the art. They are suitable for cosmetic use. In particular the compositions are suitable for application to a site of interest that has an aging-related condition or disorder. Application of the cosmetic compositions will combat these conditions thereby restoring a more youthful appearance. In addition, certain of these cosmetic compositions can be used to prevent the onset of the aging-related condition or disorder.

OIL-IN-WATER EMULSION

| Ingredient | % w/w |
|---|---|
| Water | QS |
| Xanthan Gum | 0-0.5% |
| Squalane | 0-5.00% |
| Neopentyl Glycol | 0-5.00% |
| Dicaprylate/Dicaprate | |
| PEG-8 Beeswax | 0-5.00% |
| Cetyl Alcohol | 0-5.00% |
| Glyceryl Stearate | 0-5.00% |
| Steareth-21 | 0-5.00% |
| Behenyl Alcohol | 0-5.00% |
| Butyrospermum Parkii | 0-10.00% |
| (Shea Butter) | |
| Dimethicone | 0-5.00% |
| VP/Eicosene Copolymer | 0-5.00% |
| Stearyl Alcohol | 0-5.00% |
| Ethylhexyl Palmitate (and) | 0-3.00% |
| Tribehenin (and) Sorbitan | |
| Isostearate (and) Palmitoyl | |
| Oligopeptide | |
| Phospholipids (and) | 0-10.00% |
| Saccharomyces Cerevisiae | |
| Extract (and) Tripeptide-29 | |
| (and) Sodium Chondroitin | |
| Sulfate (and) Hyaluronic | |
| Acid (and) Tocopherol | |
| Butylene Glycol | 0-5.00% |
| Sorbitol | 0-5.00% |
| Potassium Sorbate | 0-0.50% |
| PEG-12 Dimethicone | 0-5.00% |
| Cyclomethicone (and) Silica | 0-5.00% |
| Dimethyl Silylate (and) | |
| Butylene Glycol (and) | |
| Caprylyl Glycol (and) | |
| Phenoxyethanol (and) | |
| Hexylene Glycol (and) | |
| Sodium Hyaluronate | |
| Magnesium Ascorbyl | 0-0.5% |
| Phosphate | |
| Polyethylene | 0-5.00% |
| Polyacrylamide (and) C13-14 | 0-5.00% |
| Isoparaffin (and) | |
| Laureth-7 | |
| Glycyrrhiza Glabra | 0-10.00% |
| (Licorice) Root Extract | |
| Algae Extract | 0-10.00% |
| Butylene Glycol (and) Yeast | 0-10.00% |
| Polysaccharides | |
| HORSE CHESTNUT | 0-10.00% |
| (AESCULUS | |
| HIPPOCASTANUM) | |
| EXTRACT | |
| Dipeptide Diaminobutyroyl | 0-10.00% |
| Benzylamide Diacetate | |
| (and) Glycerin | |
| Acetyl Octapeptide-3 | 0-10.00% |
| Acetyl Hexapeptide-8 | 0-10.00% |
| Glycerin (and) Palmitoyl | 0-10.00% |
| Tripeptide-5 | |
| Glycerin (and) Water (and) | 0-10.00% |
| Butylene Glycol (and) | |
| Carbomer (and) Polysorbate | |
| 20 (and) Palmitoyl | |
| Oligopeptide (and) | |
| Palmitoyl Tetrapeptide-7 | |
| Glycerin (and) Palmitoyl | 0-10.00% |
| Dipeptide-5 | |
| Diaminobutyroyl | |
| Hydroxythreonine (and) | |
| Palmitoyl Dipeptide-5 | |
| Diaminohydroxybutyrate | |
| Phenoxyethanol (and) | 0-2.00 |
| Methylparaben (and) | |
| Ethylparaben (and) | |
| Butylparaben (and) | |
| Propylparaben (and) | |
| Isobutylparaben | |
| Flavor | 0-2.00% |
| Glycine Soja (Soybean) | 0-5.00% |
| Seed Extract and | |
| Lactobacillus/Capsicum | |
| Frutescens Fruit Extract | |
| DRACO PEPPERMINT | 0-1.00% |
| (WILD MINT) 10:01:00 | |
| AM | |
| Total | 100.00% |
| water | QS |
| panthenol | 0-5.00% |
| aloe barbadensis leaf juice | 0-2.00% |
| sodium phytate | 0-0.50% |
| allantoin | 0-2.00% |
| Methylparaben | 0-0.50% |
| glyceryl stearate (and) PEG- | 0-5.00% |
| 100 stearate | |
| C12-15 Alkyl Benzoate | 0-5.00% |
| Glyceryl Stearate | 0-5.00% |
| Persea Gratissima (Avocado) | 0-5.00% |
| Oil | |
| Isocetyl stearate | 0-5.00% |
| tocopheryl acetate | 0-5.00% |
| cetyl alcohol | 0-5.00% |
| dimethicone | 0-5.00% |
| Butyrospermum Parkii (Shea | 0-5.00% |
| Butter) | |
| Propylene Glycol Stearate | 0-5.00% |
| Caprylic/Capric Triglyceride | 0-5.00% |
| Stearoxytrimethylsilane (and) | 0-5.00% |
| Stearyl Alcohol | |
| Hydrogenated Lecithin | 0-5.00% |
| Glycerin (and) Water (and) | 0-10.00% |
| Butylene Glycol (and) | |
| Carbomer (and) Polysorbate | |
| 20 (and) Palmitoyl | |
| Oligopeptide (and) Palmitoyl | |
| Tetrapeptide-7 | |
| Acetyl Hexapeptide-8 | 0-10.00% |
| Dipeptide Diaminobutyroyl | 0-10.00% |
| Benzylamide Diacetate (and) | |
| Glycerin | |
| Glycerin (and) Palmitoyl | 0-10.00% |
| Tripeptide-5 | |
| Acetyl Octapeptide-3 | 0-10.00% |
| Water (and) Glycolic Acid | 0-10.00% |
| (and) Arginine | |
| Glycerin (and) Palmitoyl | 0-10.00% |
| Dipeptide-5 Diaminobutyroyl | |
| Hydroxythreonine (and) | |
| Palmitoyl Dipeptide-5 | |
| Diaminohydroxybutyrate | |
| Water (and) Pentylene Glycol | 0-5.00% |
| (and) Lecithin (and) Alcohol | |
| (and) Retinol (and) | |
| Polysorbate 20 | |
| Water (and) Phospholipids | 0-5.00% |
| (and) Retinyl Palmitate (and) | |
| Ascorbyl Palmitate (and) | |
| Tocopheryl Acetate | |
| Propylene Glycol (and) | 0-2.00% |
| Diazolidinyl Urea (and) | |
| Methylparaben (and) | |
| Propylparaben | |
| Sodium Hydroxide | 0-5.00% |
| Total | 100% |
| DEIONIZED WATER | QS |
| GLYCERIN | 0-10.00% |
| HISTIDINE HCL | 0-0.50% |
| DL PANTHENOL | 0-5.00% |
| METHYLPARABEN | 0-0.50% |
| PROTAMATE 4400 DPS | 0-5.00% |
| FINSOLV TN | 0-5.00% |
| GMS 450 | 0-5.00% |
| AVOCADO OIL | 0-5.00% |
| ISOCETYL STEARATE | 0-5.00% |
| (DERMOL ICS) | |
| VITAMIN E ACETATE | 0-5.00% |
| CETYL ALCOHOL | 0-5.00% |
| DC 200/100 | 0-5.00% |
| LANOLIN OIL | 0-5.00% |
| PGMS | 0-5.00% |
| SILICONE 580 WAX | 0-5.00% |
| VITAMIN A PALMITATE | 0-5.00% |
| BISABOLOL | 0-5.00% |
| LANOLIN ALCOHOL | 0-5.00% |
| PROPYLPARABEN | 0-0.50% |
| GERMALL II | 0-0.50% |
| MARINE COMPLEX/ | 0-5.00% |
| COLLASSOL M /CRODA | |
| BIOGEN SEA ACID CMPLX | 0-5.00% |
| SYN COLL | 0-10.00% |
| ARGIRELINE | 0-10.00% |
| MATRIXYL 3000 | 0-10.00% |
| BG-LIPOVIT A, C, E | 0-5.00% |
| HYDROLYZED ELASTIN | 0-5.00% |
| ALGAE EXTRACT | 0-5.00% |
| ECHINACEA | 0-5.00% |
| GREEN TEA EXTRACT | 0-5.00% |
| WHITE TEA EXTRACT | 0-5.00% |
| GINKGO BILOBA | 0-5.00% |
| GRAPE SEED EXTRACT | 0-5.00% |
| SODIUM ASCORBYL | 0-5.00% |
| PHOSPHATE | |
| SODIUM HYALURONATE | 0-5.00% |
| AJIDEW N-50 | 0-5.00% |
| GOTU KOLA EXTRACT | 0-5.00% |
| HYDROTRITICUM WAA | 0-5.00% |
| RETINYL PALMITATE | 0-5.00% |
| ASCORBOSILANE C | 0-5.00% |
| TOCOPHERYL LINOLEATE | 0-5.00% |
| ACTIPHYTE OF FENNEL | 0-5.00% |
| SEED | |
| EMERSOL 315 (LINOLEIC | 0-5.00% |
| ACID) | |
| EMERSOL 221 (OLIEC | 0-5.00% |
| ACID) | |
| PANSY EXTRACT | 0-5.00% |
| ROSE HIP OIL | 0-5.00% |
| BIOCELL SOD | 0-5.00% |
| CARRUBBA AROMATIC | 0-5.00% |
| LEMON G0839 | |
| Total | 100% |
| DI WATER | QS |
| DISODIUM EDTA | 0-0.50% |
| ALLANTOIN | 0-5.00% |
| DL-PANTHENOL | 0-5.00% |
| CARBOPOL 940 | 0-2.00% |
| EMCOL E607 | 0-2.00% |
| SHEA BUTTER | 0-5.00% |
| DC 200 FLUID | 0-5.00% |
| SQUALANE | 0-5.00% |
| WHITE BEESWAX | 0-5.00% |
| PGMS | 0-5.00% |
| NEOBEE M5 | 0-5.00% |
| CETYL ALCOHOL | 0-5.00% |
| GMS 450 | 0-5.00% |
| CERAMIDES III | 0-1.00% |
| CREMOPHOR RH 40 | 0-5.00% |
| (BASF) | |
| EMERSOL 315 | 0-5.00% |
| OLEIC ACID (EMERSOL | 0-5.00% |
| 221) | |
| VITAMIN A PALMITATE | 0-5.00% |
| ASORBOSILANE C | 0-5.00% |
| VITAMIN E ACETATE | 0-5.00% |
| TEA | 0-2.00% |
| MATRIXYL 3000 | 0-10.00% |
| ALOE VERA GEL | 0-5.00% |
| BIOGEN SEA COMPLEX | 0-5.00% |
| COLLASOL M | 0-5.00% |
| AVOCADO OIL | 0-5.00% |
| ROSE HIP OIL | 0-5.00% |
| PANSY EXTRACT | 0-5.00% |
| FENNEL SEED EXTRACT | 0-5.00% |
| ASCOBIC ACID | 0-5.00% |
| GREEN TEA EXTRACT | 0-5.00% |
| WHITE TEA EXTRACT | 0-5.00% |
| ECHINACEA EXTRACT | 0-5.00% |
| ALGAE EXTRACT | 0-5.00% |
| HYDROLYZED ELASTIN | 0-5.00% |
| SYN COLL | 0-10.00% |
| ARGIRELINE | 0-10.00% |
| SODIUM HYALURONATE | 0-5.00% |
| AJIDEW N 50 | 0-5.00% |
| HYDROTRITICUM WAA | 0-5.00% |
| DOWICIL 200 | 0-1.00% |
| GERMABEN II | 0-2.00% |
| BIOCELL SOD | 0-5.00% |
| SEPIGEL 305 | 0-3.00% |
| Total | 100% |
| Water | QS |
| Acrylates/C10-30 Alkyl | 0-2.00% |
| Acrylate Crosspolymer | |
| Propylene Glycol | 0-5.00% |
| Allantoin | 0-5.00% |
| Caprylic/Capric Triglyceride | 0-5.00% |
| C12-15 Alkyl Benzoate | 0-5.00% |
| Glyceryl Stearate, PEG-100 | 0-5.00% |
| Stearate | |
| Cetyl Alcohol | 0-5.00% |
| Dimethicone | 0-5.00% |
| Stearic Acid | 0-5.00% |
| Octisalate | 0-5.00% |
| Octinoate | 0-7.50% |
| Oxylbenzone | 0-4.00% |
| Titanium Dioxide, Dimethicone | 0-1.00% |
| Triethanolamine | 0-2.00% |
| Water, Glycerin, Dipeptide | 0-10.00% |
| Diaminobutyroyl Beuzylamide | |
| Diacetate | |
| Acetyl Glutmyl Heptapeptide-3 | 0-10.00% |
| Acetyl Hexapeptide-8 | 0-10.00% |
| Palmitoyl Tripeptide-5, | 0-10.00% |
| Glycerine | |
| Glycerin, Water, Butylene | 0-10.00% |
| Glycol, Carbomer, Poltsorbate | |
| 20, Palmitoyl Oligopeptide, | |
| Palmitoyl Tetrapeptide-3 | |
| Glycerin, Palmitoyl Dipeptide-5 | 0-10.00% |
| Diaminobutyoloyl | |
| Hydroxythreonine, Palitoyl | |
| Dipeptide-6 | |
| Diaminohydroxybutyrate | |
| Water, Phospholipids, | 0-5.00% |
| Tocopheryl Acetate, Retinyl | |
| Palmitate, Ascorbyl Palmitate. | |
| Essential Rose Water C5799 | 0-20.00% |
| Lemon Ginger Aromatic G0839 | 0-5.00% |
| Vanilla Essential Oil 6500170 | 0-5.00% |
| Capryloylhydroxamic Acid, | 0-5.00% |
| Caprylyl Glycol, Glycerin | |
| Total | 100% |

HYDRO-GEL

| Ingredient | % w/w |
|---|---|
| Water | QS |
| Carbomer | 0-2.00% |
| Panthenol | 0-5.00% |
| Butylene Glycol | 0-10.00% |
| Xanthan Gum | 0-2.00% |
| Bis-PEG-18 Methyl Ether | 0-5.00% |
| Dimethyl Silane | |
| Dimethicone PEG-7 Phosphate | 0-5.00% |
| Glycerin (and) Water (and) | 0-10.00% |
| Butylene Glycol (and) Carbomer | |
| (and) Polysorbate 20 (and) | |
| Palmitoyl Oligopeptide (and) | |
| Palmitoyl Tetrapeptide-7 | |
| Squalane | 0-5.00% |
| PEG-75 | 0-5.00% |
| Bisabolol | 0-5.00% |
| Oleth-2 | 0-5.00% |
| Oleth-5 | 0-5.00% |
| Trietholamine | 0-2.00% |
| Water (and) Phospholipids (and) | 0-5.00% |
| Retinyl Palmitate (and) | |
| Tocopheryl Acetate (and) | |
| Ascorbyl Palmitate | |
| Sodium Hyaluronate | 0-5.00% |
| Water (and) Glycerin (and) | 0-10.00% |
| Dipeptide Diaminobutyroyl | |
| Benzylamide Diacetate | |
| Water (and) Acetyl Glutamyl | 0-10.00% |
| Heptapeptide-1 | |
| Water (and) Acetyl Hexapeptide-8 | 0-10.00% |
| Glycerin (and) Palmitoyl | 0-10.00% |
| Tripeptide-5 | |
| Propylene Glycol (and) | 0-2.00% |
| Diazolidinyl Urea, | |
| Methylparaben, Propylparaben | |
| Hydrogenated Soybean Oil (and) | 0-5.00% |
| Vanilla Planifolia Fruit Extract | |
| Caprylic/Capric Triglyceride | 0-5.00% |
| (and) Citrus Medica Limonum | |
| (Lemon) Peel Extract (and) | |
| Zingiber Officinale (Ginger) | |
| Root Extract | |
| Rosa Centifolia Flower Water | 0-20.00% |
| Red 33 | 0-0.50% |
| Yellow 5 | 0-0.50% |
| Total | 100.00% |
| Water | QS |
| Acrylates/C10-C29 Alkyl | 0-2.00% |
| Acrylate Crosspolymer | |
| Aloe Barbadensis Leaf Juice | 0-2.00% |
| C4-24 Alkyl | 0-10.00% |
| Dimethicone/divinyldimethicone | |
| crosspolymer cyclomethicone | |
| Dimethicone/Divinyldimethicone | 0-5.00% |
| Crosspolymer Dimethicone | |
| C4-24 Alkyl Dimethicone/ | 0-5.00% |
| Divinyldimethicone | |
| Crosspolymer Isononanoate | |
| RESSASSOL VS | 0-5.00% |
| Sodium Hyaluronate | 0-15.00% |
| Panthenol | 0-5.00% |
| Pentavitin | 0-5.00% |
| ADVANCED MOISTURE | 0-5.00% |
| COMPLEX | |
| Sodium Cocoyl Amino | 0-5.00% |
| Acids, Sarcosine, Potassium | |
| Aspartate, Magnesium Asparate | |
| Water, Mannan | 0-5.00% |
| BROOKOSOME ACE | 0-5.00% |
| Acetyl hexapeptide-8 | 0-10.00% |
| glycerine, Palmitoyl Tripeptide-5 | 0-10.00% |
| LIPOSOME ACE | 0-5.00% |
| Prunus Amygdalus Dulis (Sweet | 0-5.00% |
| Almond) Oil | |
| Glycosamines | 0-5.00% |
| Caprylyl Glycol (and) | 0-5.00% |
| Phenoxyethanol | |
| Sodium Hydroxide | 0-2.00% |
| Total | 100.00% |
| DEIONIZED WATER | QS |
| CARBOPOL 940 | 0-2.00% |
| BUTYLENE GLYCOL | 0-5.00% |
| ALOE 200M | 0-2.00% |
| GLYCERIN | 0-5.00% |
| KESSCO OP | 0-20.00% |
| ALMOND OIL | 0-10.00% |
| VITAMIN E ACETATE | 0-5.00% |
| PROCOL CS-20D | 0-5.00% |
| CETYL ALCOHOL | 0-5.00% |
| BISABOLOL | 0-5.00% |
| MATRIXYL 3000 | 0-10.00% |
| TEA | 0-2.00% |
| GERMABEN | 0-2.00% |
| LIPOSOME ACE | 0-5.00% |
| POMEGRANATE | 0-5.00% |
| BIOFERMENT | |
| SEPPI-CALM S | 0-5.00% |
| ARGIRELINE | 0-10.00% |
| SYN-COLL | 0-10.00% |
| KONJAC MANNAN | 0-5.00% |
| Total | 100.00% |
| Water | QS |
| Hydroxyethylcellulose | 0-2.00% |
| Xanthan Gum (and) Pectin (and) | 0-10.00% |
| Hydrolyzed Vegetable Protein | |
| (and) Serine (and) Arginine | |
| (and) Proline | |
| Glycerin (and) Palmitoyl | 0-10.00% |
| Dipeptide-5 Diaminobutyroyl | |
| Hydroxythreonine (and) | |
| Palmitoyl Dipeptide-5 | |
| Diaminohydroxybutyrate | |
| Water (Aqua) (and) | 0-10.00% |
| Pseudoalteromonas Ferment | |
| Extract (and) Hydrolyzed Wheat | |
| Protein (and) Hydrolyzed Soy | |
| Protein (and) Tripeptide-10 | |
| Citrulline (and) Tripeptide-1 | |
| (and) Lecithin (and) | |
| Xanthan Gum (and) Carbomer | |
| (and) Triethanolamine (and) | |
| Phenoxyethanol (and) Butylene | |
| Glycol (and) Caprylyl Glycol | |
| Lactobacillus/Punica Granatum | 0-5.00% |
| Fruit Ferment Extract | |
| Algae Extract, Pullulan. | 0-5.00% |
| Glycerin (and) Water (and) | 0-10.00% |
| Butylene Glycol (and) Carbomer | |
| (and) Polysorbate 20 (and) | |
| Palmitoyl Oligopeptide (and) | |
| Palmitoyl Tetrapeptide-7 | |
| Carnosine | 0-1.00% |
| Glycerin (and) Palmitoyl | 0-10.00% |
| Tripeptide-5 | |
| Water (and) Phospholipids (and) | 0-5.00% |
| Retinyl Palmitate (and) Ascorbyl | |
| Palmitate (and) Tocopheryl | |
| Acetate | |
| Mica | 0-1.00% |
| Benzophenone-3 | 0-3.00% |
| Pentylene Glycol | 0-5.00% |
| Lipolight OAP | 0-5.00% |
| Disodium EDTA | 0-0.50% |
| Phenoxyethanol (and) Caprylyl | 0-2.00% |
| Glycol (and) Ethylhexylglycerin | |
| (and) Hexylene Glycol | |
| Water (Aqua) (and) | 0-10.00% |
| Hexapeptide-10 | |
| PEG-8 Dimethicone | 0-5.00% |
| Sensitive Skin Maskant 905613 | 0-2.00% |
| Lactobacillus/Eriodictyon | 0-5.00% |
| Californicum Ferment Extract | |
| Ethanol | 0-5.00% |
| Phenylethyl Resorcinol | 0-5.00% |
| Citric Acid | 0-2.00% |
| Total | 100% |
| Water | QS |
| Carbomer | 0-2.00% |
| Methyl Gluceth-20 | 0-5.00% |
| Allantoin | 0-5.00% |
| Aloe Barbadensis Leaf Juice | 0-5.00% |
| Disodium EDTA | 0-0.50 |
| Glycerin (and) Water (and). | 0-10.00% |
| Sodium Polyacrylate | |
| Trietholamine | 0-2.00% |
| C12-15 Alkyl Benzoate | 0-5.00% |
| Glycerin (and) Water (and) | 0-10.00% |
| Butylene Glycol (and) Carbomer | |
| (and) Polysorbate 20 (and) | |
| Palmitoyl Oligopeptide (and) | |
| Palmitoyl Tetrapeptide-7 | |
| Cyclomethicone | 0-5.00% |
| Dimethicone | 0-5.00% |
| Prunus Dulcis (Almond) Oil | 0-5.00% |
| Polysorbate 20 | 0-5.00% |
| PEG-12 Dimethicone | 0-5.00% |
| Tocopheryl Acetate | 0-5.00% |
| Retinyl Palmitate | 0-5.00% |
| Water (and) Glycerin (and) | 0-10.00% |
| Dipeptide Diaminobutyroyl | |
| Benzylamide Diacetate | |
| Water (and) Acetyl Glutamyl | 0-10.00% |
| Heptapeptide-1 | |
| Water (and) Acetyl Hexapeptide-8 | 0-10.00% |
| Glycerin (and) Palmitoyl | 0-10.00% |
| Tripeptide-5 | |
| Cyclomethicone (and) Silica | 0-5.00% |
| Dimethyl Silylate (and) Butylene | |
| Glycol (and) Caprylyl Glycol | |
| (and) Phenoxyethanol (and) | |
| Hexylene Glycol (and) Sodium | |
| Hyaluronate | |
| Water (and) Prunus Amygdalus | 0-5.00% |
| Dulcis (Sweet Almond) Seed | |
| Extract | |
| Boron Nitride | 0-2.00% |
| Phenoxyethanol (and) | 0-2.00% |
| Methylparaben (and) Ethylparaben | |
| (and) Butylparaben (and) | |
| Propylparaben (and) Isobutylparab | |
| Total | 100.00% |

AQUEOUS SERUM

| Ingredient | % w/w |
|---|---|
| Water | QS |
| AH CARE SOLUTION | 0-10.00% |
| Chamomile Extract | 0-5.00% |
| Green Tea Extract | 0-5.00% |
| Comfrey Extract | 0-5.00% |
| Butylene Glycol | 0-5.00% |
| Aloe Vera Gel | 0-5.00% |
| Sodiuim Lactate Solution (60%) | 0-10.00% |
| SALICYLIC ACID | 0-0.49% |
| L-ARGININE | 0-5.00% |
| P-ALANINE | 0-5.00% |
| GABA | 0-5.00% |
| Procetyl AWS | 0-5.00% |
| GammaLinoleinic Acid | 0-5.00% |
| Ethoxydiglycol | 0-5.00% |
| Lemon Ginger Aromatic | 0-5.00% |
| Germaben II | 0-2.00% |
| Vitamin E | 0-5.00% |
| Vitamin C (Stay C-50) | 0-5.00% |
| Vitamin A Palmitate 1.0 m | 0-5.00% |
| I.U./g and Vitamin D3 100,000 | |
| I.U./g | |
| ROSE WATER | 0-10.00% |
| Witch Hazel | 0-10.00% |
| Sodium Hydroxide 50% soln. | 0-7.00% |

### Example 2

A composition that can be topically applied by a human as an anti-cellulite cream emulsion includes the following ingredients in three phases:

| | | concentration | |
|---|---|---|---|
| phase | component | (by weight) | function and source |
| water-based phase | water | 50-70% | skin moisturizer |
| water-based phase | Carbopol 940 polymer (an | 1-5% | formula stabilizer (Noveon, Inc., |
| | acrylic acid copolymer | | Ohio) |
| | referred to as Carbomer and | | |
| | with CAS No. 9007-17-4) | | |
| oil-based phase | cetearyl alcohol | 1-5% | moisturizer (Chemron, Inc.) |
| oil-based phase | Ceteareth-20 | 2-4% | emulsifier (Chemron, Inc.) |
| oil-based phase | vitamins A, C, E | 1-3% | oil/water soluble vitamins (DSM |
| | | | Nutritional Divison, USA) |
| oil-based phase | avocado oil | 2-4% | skin emollient (Arista Oils, Inc.) |
| peptide phase | ARGIRELINE [acetyl | 1-5% | inhibits SNARE complex |
| | hexapeptide-3 (TIMP)] | | formation and catecholamine |
| | | | release (Lipotec S.A., Spain) |
| peptide phase | SYN-COLL (palmiotyl | 2-6%, for | mimics tissue growth factor |
| | tripeptide-3 in a water-and | instance, 3% | (Pentapharm, Inc., Basel, |
| | glycerine-based solution) | | Switzerland) |
| peptide phase | MATRIXYL 3000 (palmitoyl | 1-3% | TIMP & tissue growth (Sederma, |
| | oligopeptide blended with | | Inc., France) |
| | tetrapeptide-1) | | |
| peptide phase | SYN-AKE [glycerine, water | 1.5-7% | tripeptide muscle relaxant |
| | and a synthetic tripeptide | | (Pentapharm, Inc., Basel, |
| | (dipeptide diaminobutyroyl | | Switzerland) |
| | benzylamide diacatate)] | | |
| peptide phase | SNAP-8 solution (water and | 2-6% | prevents neuro-transmitter release |
| | acetyl glutamyl heptapeptide- | | and muscle contraction (Lipotec |
| | 1) | | Inc., S.A.) |

### Manufacturing Procedure:

The water phase and the oil phase are heated to 55° C. The oil phase is then added to the water phase with high-energy mixing (homomixing). The mixture is then sweep cooled to 35° C., and the peptide phase is added, and mixing is continued, while cooling to 30° C.

In a test group of three panels, each having twenty users, samples with compositions having the above-cited water-phase and oil-phase components at concentrations in the above ranges were used (a) without the peptide phase (control) phase, (b) with 3 of the 5 peptide-phase ingredients (partial) and (c) with all 5 peptide ingredients (complete) during a six week period. Significant positive performance scores (versus existing brands) were evident in the complete formulation after two weeks of daily usage. Significant scores with the partial formulation were recorded after 4 weeks of daily usage. The control samples showed no specific advantages versus the existing brands in the six-week panel test. Important attributes that scored well for both the partial and complete compositions included:

A) improved skin tightness,

B) reduction in skin wrinkles,

C) reduction in skin bumps,

D) more supple skin, and

E) enhanced skin elasticity.

### Example 3

Another formulation, which was balanced for moisturizing anti-aging, wrinkle-reduction benefits, has the following composition:

| | | concentration | |
|---|---|---|---|
| phase | component | (by weight) | function and source |
| water-based phase | water | 40-85% | skin moisturizer |
| water-based phase | vitamin C (ascorbic acid) | 1-3% | (DSM Nutritional, Inc.) |
| water-based phase | sodium hyaluronate | 2-5% | moisturizer (Rita |
| | | | Chemical, Inc.) |
| water-based phase | Natrosol 250 HR | 0.5-2.0 | thickener (Hercules, Inc.) |
| oil-based phase | Myrj 52 (PEG-40 Searate) | 1-3% | emulsifier (Uniqema Inc.) |
| oil-based phase | Brij 52 (Ceteth-2) | 0.5-2.5% | emulsifier (Uniqema Inc.) |
| oil-based phase | squalene | 2.0-5.0% | skin moisturizer (Arista |
| | | | Oils, Inc.) |
| oil-based phase | glyceryl monostearate | 3.0-5.0% | skin moisturizer (Lipo |
| | | | Chemicals, Inc.) |
| oil-based phase | apricot oil | 4-6% | skin emollient (Arista Oils, |
| | | | Inc.) |
| peptide phase | ARGIRELINE [acetyl | 2-10% | inhibits SNARE complex |
| | hexapeptide-3 (TIMP)] | | formation and |
| | | | catecholamine release |
| | | | (Lipotec, Inc., S.A. Spain) |
| peptide phase | SYN-COLL (palmitoyl | 2-6%, for | mimics tissue growth |
| | tripeptide-3 in a water-and | instance, 3% | factor (Pentapharm, Inc., |
| | glycerine-based solution) | | Basel, Switzerland) |
| peptide phase | MATRIXYL 3000 | 1-3% | TIMP & tissue growth |
| | (palmitoyl oligopeptide | | (Sederma, Inc., France) |
| | blended with tetrapeptide-1) | | |
| peptide phase | SYN-AKE [glycerine, water | 2.5-8% | muscle relaxant |
| | and a synthetic tripeptide | | (Pentapharm, Inc., Basel, |
| | (dipeptide diaminobutyroyl | | Switzerland) |
| | benzylamide diacatate)] | | |
| peptide phase | SNAP-8 solution (water and | 2-6% | prevents neuro-transmitter |
| | acetyl glutamyl | | release and muscle |
| | heptapeptide-1) | | contraction (Lipotec Inc., |
| | | | S.A.) |

### Manufacturing Procedure:

The water and oil phases are heated in separate kettles to 70° C. The oil phase is then added to the water phase with vigorous agitation (homomixing). After 5 minutes, the mixture is sweep cooled to 35° C. The peptide phase is then added to the cooled mixture, and mixing is continued to 30° C.

### Panel Testing:

In a series of panels (with fifteen members per panel), three blind coded formulations were tested. These formulations comprised the above-cited water-phase and oil-phase components at concentrations in the above ranges. The formulations included a control formulation (no peptides), a partial formulation (3-4 of the peptides listed), and a full formulation (all of the peptides listed). Each panel member was instructed to use her best wrinkle remover on one side of her face and the test product on the other each day for six weeks. Weekly interviews were made to record their observations (if any). After three weeks, directional significant preferences were reported from the panel using the complete formulation. These preferences became significant (versus their brand) during the 4th week. The partial-formulation panel began reporting directional significant benefits at the end of the 5th and at the beginning of the 6th week of testing. The control-product panelists felt their brands outperformed the control product. Test-product attributes that were recorded included: skin tightening, return of skin elasticity, skin smoothness, healthier appearance, supple feel in eye skin, reduction of wrinkle appearance, and elimination of dark circles.

In describing embodiments of the disclosure, specific terminology is used for the sake of clarity. For purposes of description, each specific term is intended to at least include all technical and functional equivalents that operate in a similar manner to accomplish a similar purpose. Additionally, in some instances where a particular embodiment of the disclosure includes a plurality of elements or method steps, those elements or steps may be replaced with a single element or step; likewise, a single element or step may be replaced with a plurality of elements or steps that serve the same purpose. Moreover, while this disclosure has been shown and described with references to particular embodiments thereof, those skilled in the art will understand that various other changes in form and details may be made therein without departing from the scope of the disclosure.

All publications and patent applications cited in this specification are herein incorporated by reference as if each individual publication or patent application were specifically and individually indicated to be incorporated by reference.

Although the foregoing disclosure has been described in some detail by way of illustration and example for purposes of clarity of understanding, it will be readily apparent to those of ordinary skill in the art in light of the teachings of this disclosure that certain changes and modifications may be made thereto without departing from the spirit or scope of the appended claims.

What is claimed is:

1. A cosmetic composition for topical application to the skin, comprising as active agents:
a. acetyl hexpeptide;
b. palmitoyl tripeptide-3;
c. palmitoyl oligopeptide;
d. palmitoyl tetrapeptide-7
e. dipeptide diaminobutyroyl benzylamide diacatate;
f. acetyl glutamyl heptapeptide-1; and a cosmetically acceptable vehicle.

2. The cosmetic composition of claim 1, wherein the:
a. acetyl hexpeptide is present in an amount that ranges from about 0.001 to about 20% by weight;
b. palmitoyl tripeptide-3 is present in an amount that ranges from about 0.001 to about 20% by weight;
c. dipeptide diaminobutyroyl benzylamide diacatate is present in an amount that ranges from about 0.001 to about 20% by weight;
d. acetyl glutamyl heptapeptide-1 is present in an amount that ranges from about 0.001 to about 20% by weight; and
e. palmitoyl oligopeptide and palmitoyl tetrapeptide-7 is present in an amount that ranges from about 0.001 to about 20% by weight.

3. The cosmetic composition of claim 1, further comprising a microsponge, a microsphere, a liposome or a micelle.

4. The cosmetic composition of claim 1, further comprising a lipophilic agent.

5. The cosmetic composition of claim 1, further comprising a surfactant.

6. The cosmetic composition of claim 1, wherein the pH of said composition is in the range of about a 3 to about a 7.

7. The cosmetic composition of claim 1, wherein the pH of said composition is in the range of about a 4.5 to about a 6.5.

8. The cosmetic composition of claim 1, wherein said composition is formulated for application to the skin of the face; surrounding the eye; lips; surrounding the lips; forehead;
neck; hands; fingers; feet; toes; skin of the joints; under arms; and body.

9. The cosmetic composition of claim 1, wherein said composition is formulated for application to the skin surrounding the eyes, mouth, lips, nose, forehead, ears, neck, hands, fingers, feet, toes, and overall body.

10. The composition of claim 1, wherein said cosmetically acceptable vehicle is an oil in water, or water in oil emulsion.

11. The composition of claim 1, wherein said cosmetically acceptable carrier is a member selected from the group consisting of glycerin, butylene glycol, propylene glycol, water, jojoba oil, sweet almond oil, soybean oil, sunflower oil, and apricot oil.

12. The composition of claim 1, where the composition does not include one or more of a paraben, a preservative, a petro chemical, a synthetic dye, or a sulfate.

13. The composition of 1, wherein said composition further comprises a sunblock.

14. A method for conditioning the skin, comprising:
applying topically a cosmetic composition comprising:
a. acetyl hexpeptide;
b. palmitoyl tripeptide-3;
c. palmitoyl oligopeptide;
d. palmitoyl tetrapeptide-7
e. dipeptide diaminobutyroyl benzylamide diacatate;
f. acetyl glutamyl heptapeptide-1; and a cosmetically acceptable vehicle.

15. The method of claim 14, wherein the composition comprises:
a. acetyl hexpeptide is present in an amount that ranges from about 0.001 to about 20% by weight;
b. palmitoyl tripeptide-3 is present in an amount that ranges from about 0.001 to about 20% by weight;
c. dipeptide diaminobutyroyl benzylamide diacatate is present in an amount that ranges from about 0.001 to about 20% by weight;
d. acetyl glutamyl heptapeptide-1 is present in an amount that ranges from about 0.001 to about 20% by weight; and
e. palmitoyl oligopeptide and palmitoyl tetrapeptide-7 is present in an amount that ranges from about 0.001 to about 20% by weight.

16. The method of claim 14, wherein the composition further comprises a microsponge, a microsphere, a liposome or a micelle.

17. The method of claim 14, wherein the composition further comprises a microsponge.

18. The method of claim 14, wherein the composition further comprises a lipophilic agent.

19. The method of claim 14, wherein the composition further comprises a surfactant.

20. The method of claim 14, further comprising applying said composition to the skin around the eyes, mouth, lips, eyelids, eyebrows, nose, ears, neck, hands, feet, fingers, toes, and overall body.
